(19) United States
(12) Patent Application Publication
(10) Pub. No.: US 2026/0033533 A1
(43) Pub. Date: Feb. 5, 2026

(54) SMALL STEP SIZE AND HIGH RESOLUTION AEROSOL GENERATION SYSTEM AND METHOD

(71) Applicant: PNEUMA RESPIRATORY, INC., Boone, NC (US)

(72) Inventors: Gregory Rapp, Boone, NC (US); Jeffrey Miller, Boone, NC (US); Shi-Bo Wang, Boone, NC (US); Judson Sidney Clements, Boone, NC (US)

(73) Assignee: PNEUMA RESPIRATORY, INC., Boone, NC (US)

(21) Appl. No.: 18/997,390

(22) PCT Filed: Jul. 18, 2023
(86) PCT No.: PCT/US2023/028043

§ 371 Date: Jan. 21, 2025

Related U.S. Application Data
(60) Provisional application No. 63/390,209, filed on Jul. 18, 2022.

(51) Int. Cl. A24F 40/05; A24F 40/10; A24F 40/42; A24F 40/50; A61M 11/00; B05B 17/06

(52) U.S. Cl. CPC A24F 40/05; A24F 40/10; A24F 40/42; A24F 40/50; A61M 11/005; B05B 17/0669

(57) ABSTRACT

An aerosol generation device includes a circuit or microcontroller for detecting and adjusting vibration frequency of an electronic transducer to a determined resonance frequency that changes during operation of the device.

### CROSS-REFERENCE TO RELATED APPLICATIONS

This application claims the benefit of priority of U.S. Provisional Patent Application No. 63/390,209 filed Jul. 18, 2022, which is incorporated herein by reference in its entirety.

### FIELD

The present disclosure relates generally to aerosol generation devices that include a vibrating transducer, such as piezoelectric transducer. Examples include droplet delivery devices that deliver fluids that are inhaled into the mouth, throat, nose, and/or lungs.

### BACKGROUND

Aerosol generation devices may include a vibrating transducer, such as a piezoelectric transducer, to create aerosolized droplets for a variety of applications. Some aerosol generation devices include droplet delivery systems directed to both therapeutic and non-therapeutic uses. Current droplet delivery systems include a variety of inhaler type systems. Some examples are metered dose inhalers (MDI), pressurized metered dose inhalers (p-MDI), pneumatic devices, and ultrasonic-driven devices. Such droplet delivery systems are directed to both therapeutic and non-therapeutic uses and may include mouthpieces and nosepieces to provide for inhalation of the fluid droplets.

### SUMMARY OF THE INVENTION

In one embodiment of the invention, an aerosol generation device comprises: a reservoir configured to supply a volume of fluid; a mesh in fluid communication with the reservoir and operably coupled to a vibrating member; an electronic transducer coupled to the vibrating member, the electronic transducer configured to operate at a first frequency that oscillates the vibrating member to generate an ejected stream of droplets through the mesh; and a circuit board or microcontroller providing a plurality of tuning signal waves to the electronic transducer during a tuning mode and measuring a current provided at a constant voltage; and wherein the circuit board or microcontroller determines a detected resonance frequency at which a highest current draw is detected and selects the detected resonance frequency at the highest current draw to drive the electronic transducer. In embodiments, the electronic transducer is a piezoelectric transducer.

In a further embodiment, an aerosol generation device of the invention includes a membrane coupled between the vibrating member and mesh.

In a further embodiment, an aerosol generation device of the invention is a droplet delivery device including a mouthpiece or nosepiece configured for inhalation.

In a further embodiment, an aerosol generation device of the invention includes the detected resonance frequency having a lower step size than the first frequency.

In another embodiment, an aerosol generation device of the invention includes a piezoelectric transducer operable to generate a droplet from fluid in the device; an ejector mechanism coupled to the piezoelectric transducer and configured to receive the fluid; and an auto-tuning circuit or microcontroller configured to adjust a first driving frequency of the piezoelectric transducer to a second driving frequency of the piezoelectric transducer to compensate for resonant frequency drift.

The aerosol generation in a further embodiment further includes the auto-tuning or microcontroller configured to conduct current analysis on the piezoelectric transducer during operation and providing adjustment to the second driving frequency based on measurement of a highest current draw across various frequencies tested during the current analysis.

In embodiments of the invention, the ejector mechanism includes an aperture plate coupled to the piezoelectric transducer.

In further embodiments, the second driving frequency has a lower step size than the first driving frequency.

In another embodiment of the invention, an aerosol generation device includes a reservoir configured to supply a volume of fluid to a mesh of an ejector mechanism, an electronic transducer operable to operate at a first frequency based upon a signal wave generated by a circuit, wherein the signal wave has a step size smaller than about 200 Hz, and wherein the transducer is operable to vibrate a mesh and generate at least one droplet from fluid supplied by the reservoir to the mesh. a microcontroller or circuit that provides a plurality of signal waves to the electronic transducer during a tuning mode and measuring a current provided at a constant voltage, determines a detected resonance frequency at which a highest current draw is detected from providing the plurality of signal waves, and selects the detected resonance frequency to drive the electronic transducer.

In embodiments of the invention, the electronic transducer is a piezoelectric transducer.

The aerosol generation device in various embodiments is a droplet delivery device including a mouthpiece or nosepiece configured for inhalation.

In embodiments, an aerosol generation device further includes a membrane coupled between the piezoelectric transducer and the aperture plate.

### BRIEF DESCRIPTION OF THE DRAWINGS

The novel features of the disclosure are set forth with particularity in the appended claims. A better understanding of the features and advantages of the present inventive concept will be obtained by reference to the following detailed description that sets forth illustrative examples, in which the principles of the disclosure are utilized, and the accompanying drawings of which:

FIG. 1 illustrates an example graph of driving a piezoelectric transducer at two frequencies to select the one with the highest current in an embodiment of the invention.

FIG. 2 illustrates an example graph of current vs. frequency for 100 Hz current profile in an embodiment of the present invention.

FIG. 3 illustrates an example graph of displacement of a piezoelectric transducer at different frequencies in an embodiment of the present invention.

FIG. 4 is a cross-sectional view of major components of a droplet delivery device in accordance with an embodiment of the disclosure.

FIG. 5 illustrates a cross-sectional view of a vibrating member enclosure of a droplet delivery device utilizing membrane-driven aerosolization in accordance with one embodiment of the disclosure.

FIG. 6A illustrates a side plan view of an exemplary aperture plate and annulus ring, in accordance with an embodiment of the disclosure.

FIG. 6B illustrates a top plan view of an exemplary aperture plate and annulus ring, in accordance with an embodiment of the disclosure.

FIG. 6C illustrates a cross-section of an exemplary aperture plate and annulus ring configuration, in accordance with an embodiment of the disclosure.

### DETAILED DESCRIPTION

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

Droplet delivery devices include an ejector mechanism with a mesh, aperture plates and like substrates having desirably sized holes and producing desirable surface contact angle that creates droplets from liquid passing through the mesh when a powered transducer acts on the liquid and ejector mechanism. In some devices a membrane may be oscillated by a powered transducer to push the liquid through the mesh and create droplets (“push mode”), while in other devices a transducer can be coupled directly to oscillate the mesh to create droplets. Examples of devices including such ejector mechanisms with substrates having apertures are described in U.S. Patent Application Pub. No. US2022/0401661 entitled “DELIVERY DEVICE WITH PUSH EJECTION” published Dec. 22, 2022, International Publication Number WO 2020/264501 entitled “DELIVERY OF SMALL DROPLETS TO THE RESPIRATORY SYSTEM VIA ELECTRONIC BREATH ACTUATED DROPLET DELIVERY DEVICE” published Dec. 30, 2020, and International Publication Number WO 2020/227717 entitled “ULTRASONIC BREATH ACTUATED RESPIRATORY DROPLET DELIVERY DEVICE AND METHODS OF USE” published Nov. 12, 2020, all of which are herein incorporated by reference in their entirety, including incorporation of such publications and patent applications as are cited and incorporated by reference or relied upon in the referenced disclosures.

The present technology can be implemented in different configurations. In one example, the present technology implements a vibrating member, such as a piezoelectric transducer with a horn (that may also be coupled to a membrane as in “push mode” technology), that pushes liquid through a fixed mesh to generate droplets. The configuration of the horn and mesh and other components can vary, but at least one example is presented herein. In another example, the present technology implements a vibrating mesh operable to move in and out of a liquid, thereby generating a jet of liquid, exiting the mesh, that turns into a droplet.

In one example, the present technology implements a piezoelectric transducer to eject a liquid through a mesh. In at least one example, the ejection of liquid results in the formation of at least one droplet.

The present technology implements a circuit having one or more clocks that can have a clock frequency. In some embodiments, the clock(s) can be further refined as needed using additional circuitry. A piezoelectric transducer has a specific resonance frequency that it runs at to have the maximum amplitude thereby maximizing the liquid ejection. Furthermore, the transducer is operable to provide the most consistent amount of liquid ejected. The circuit board is operable to generate a signal wave that is sent to the piezoelectric transducer. The signal wave is operable to drive the piezoelectric transducer to operate in a particular way. The circuit board is operable to generate a particular frequency step size to generate the signal wave. The step size is determined by the clock frequency of the circuit. In at least one example, the clock frequency can come from a microcontroller. In another example, the clock frequency can come from the circuit board. The present technology implements a high clock frequency to provide enhanced resolution in the step size. For example, the present technology can decrease an existing step size from about 400 Hz to 1 Hz. This decrease in step size allows the applied frequency to be closer to the piezoelectric transducer assembly resonance frequency which maximizes the mass ejection. The present technology can implement a small step size for increased performance.

The present technology is also operable to find and drive the piezoelectric transducer at its resonance frequency. In manufacturing and selecting different piezoelectric transducers, the resonance frequency might differ across a production run or further from one production run to another. Additionally, there are other factors that change a piezoelectric transducer's resonance frequency. For example, the resonance frequency can be changed based on the temperature of the piezoelectric transducer. Additionally, as the piezoelectric device vibrates during operation, the temperature of the piezoelectric transducer changes. Thus, during operation and ejection of the liquid, the piezoelectric transducer experiences a change in resonance frequency. Additional factors that can change the resonance frequency include the amount of liquid being ejected and type of liquid.

Electrical current analysis can compensate for the decrease in resonant frequency of a piezoelectric transducer during actuation due to rising temperature. This is done by doing a refresh at specific time intervals (e.g. 1 ms, 10 ms, 100 ms, 250 ms, 500 ms, 1000 ms). The refresh monitors the electrical current of the piezoelectric transducer actuation at a range of frequencies near the previously found resonant frequency. The driving frequency is set to a new frequency to compensate for the resonant frequency drift. It can be assumed that the resonant frequency will only decrease during actuation, so only frequencies below the initial resonant frequency should have to be monitored to effectively compensate for the resonant frequency shift.

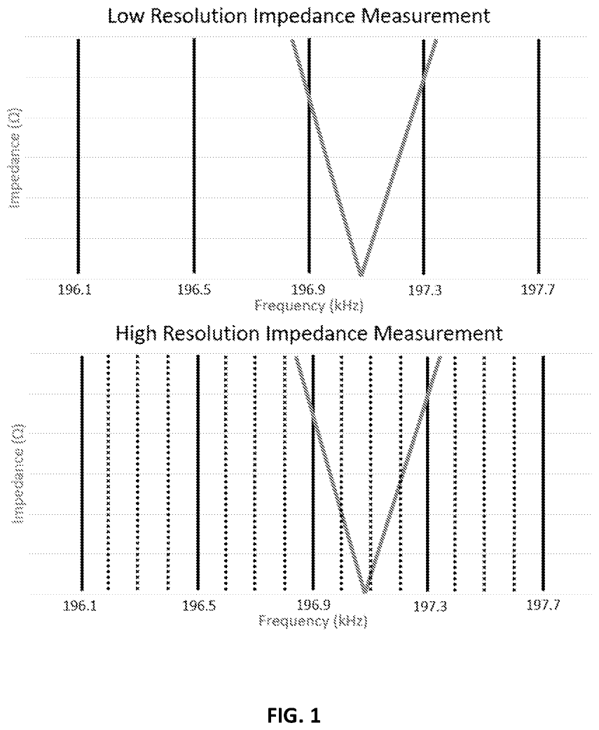

The present technology implements an auto-tune feature, whereby the circuit tries a plurality of frequencies and selects the frequency closest to the resonance frequency. In order to determine the resonance frequency, the circuit can use sensors to determine the resonance frequency. For example, the resonance frequency can be defined as the point at which impedance is the lowest. The circuit can also use a fixed voltage to drive the piezoelectric device and detect when the current is at the highest across multiple frequencies. The determination of when the current is at the highest for a particular frequency will provide the resonance frequency. For example, the auto tune mode can drive the piezoelectric transducer at a plurality of frequencies and measure the current at each frequency. The auto tune feature can then select the one with the highest current. An example of the change in driving frequency is illustrated in FIG. 1. The device can be configured to auto-tune at a predetermined time and/or based on a temperature measurement of the piezoelectric transducer. The auto-tune allows the ejection of liquid to be made at the resonance frequency thereby maximizing the amount of liquid ejected and providing the most consistent ejection.

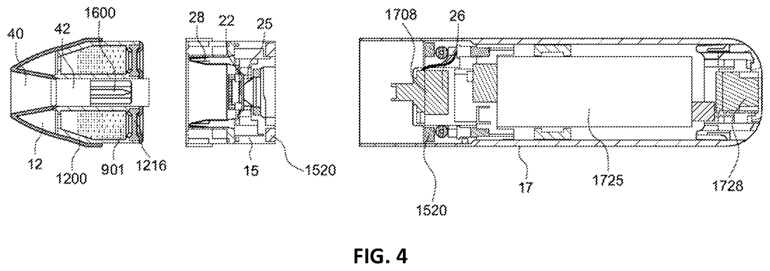

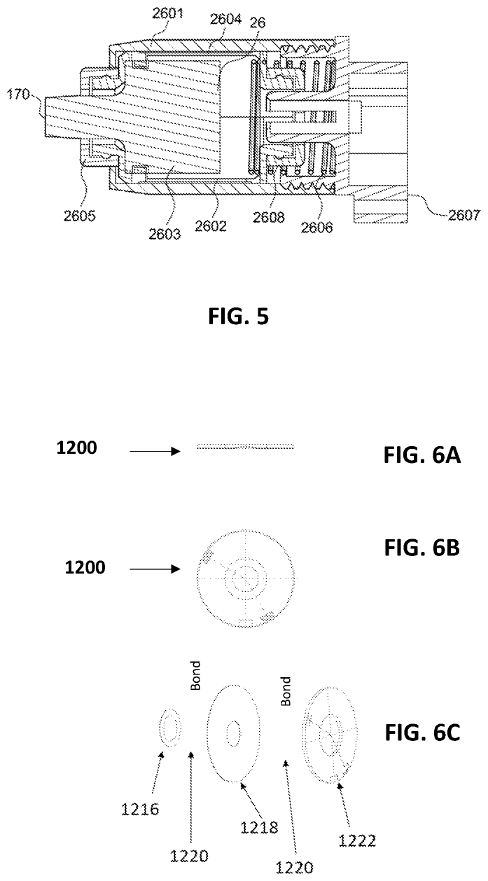

The above description applies to a configuration of the present technology that implements a piezoelectric transducer, horn, and liquid assembly, which are further described with reference to FIGS. 4 and 5. In other examples, the present technology can implement a piezoelectric transducer, mesh, and liquid assembly. The present technology implements controller, circuit board, clock, and/or microcontroller having the above features to drive the piezoelectric transducer, which then causes the mesh to vibrate. As the mesh vibrates, the fluid is expelled through the mesh in a jet. The jet turns into a droplet. Further details regarding this configuration are presented below with reference to FIGS. 6A-6C.

FIG. 1 illustrates two different step sizes that are used to drive the frequency selection. The lower resolution has a frequency variation of about 400 Hz. The higher resolution has a frequency variation of about 100 Hz. Measuring the impedance allows the resonance frequency to be determined, and it can be more closely approximated by the higher resolution. Thus, implementing the smaller step size in regard to frequency generation allows for the resonance frequency to be better approximated and matched to generate the best signal possible to control the piezoelectric transducer. In embodiments to the invention, the smaller step size is lower than about 200 Hz, and preferably at around 100 Hz, and even to a lower step size of 1 Hz in certain embodiment, such that a more precise step size can more accurately determine the resonance frequency of an electronic/piezoelectric transducer. In the described embodiment with reference to FIG. 1, the resonance frequency of a piezoelectric transducer is determined by actuating the transducer with several different frequencies and analyzing the current draw at each frequency in the system, and is not technically a full impedance analysis.

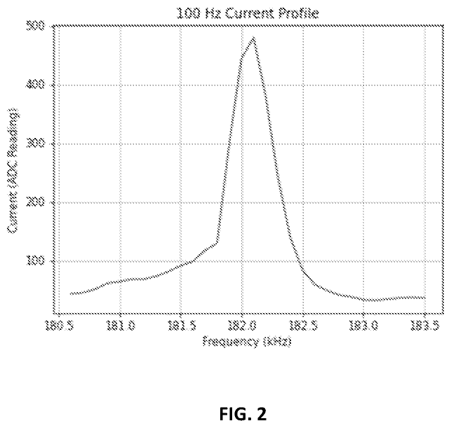

With reference to FIG. 2, the 100 Hz current profile graph shows data that was extracted from a microcontroller of an aerosol generation device of the invention with a piezoelectric transducer to verify that the resonant frequency is determinable, i.e., the frequency chosen is the peak current found in the illustrated data.

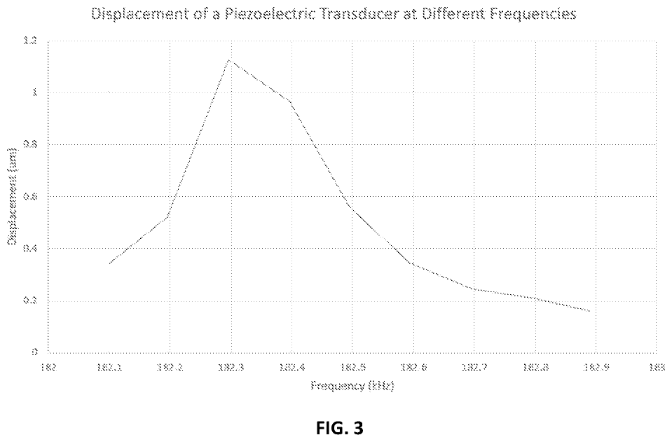

The graph of FIG. 3 illustrates piezoelectric transducer displacement at different frequencies. The data was acquired with a laser vibrometer (the MV-H™ Series vibrometer from OmniSensing Photonics, LLC (Columbia, MD)) and shows how precise frequency step sizes of 100 Hz can affect the mechanical displacement of the piezoelectric transducer. Peak to peak voltage for the measured data of FIG. 3 was about 5 V.

An example device according to the present technology can be described as follows and illustrated in FIG. 4. A droplet delivery device having a membrane that cooperates with a mesh further includes a PZT-based ultrasonic transducer coupled to a vibrating member having a tip portion made of at least one of Grade 5 titanium alloy, Grade 23 titanium alloy, and about 99% or higher purity titanium. In certain embodiments, the vibrating member's tip includes a sputtered on outer layer of and about 99% or higher purity titanium providing a smooth tip surface configured to contact an underlying bottom surface of the membrane that is opposite an exterior top surface of the membrane positioned nearest the mesh to help reduce wear of the membrane and increase the longevity and operation consistency of the membrane (and also possibly vibrating member's tip portion). The transducer can be configured to have the desired higher frequency as described above. A vibrating member 1708 and transducer 26 work in conjunction with a membrane 25 and mesh 22 to aerosolize fluid 901, which is held in a reservoir 1200 and supplied to the mesh 22 using various methods (e.g., wick material, hydrophilic coatings, capillary action, etc.). Preferably the vibrating member is coupled to the transducer, such as by bonding (e.g., adhesives and the like), welding, gluing, physical connections (e.g., brackets and other mechanical connectors), and the like. The transducer and vibrating member interact with the membrane to push fluid through the mesh. As illustrated and described in various embodiments, the membrane may in some cases contact the mesh while also “pushing” fluid through holes in the mesh and may in other cases be separated without contacting the mesh to push liquid through holes in the mesh. The transducer may comprise one or more of a variety of materials (e.g., PZT, etc.). In certain embodiments the transducer is made of lead-free piezoelectric materials to avoid creation of unwanted or toxic materials in a droplet delivery device intended for human inhalation. The vibrating member may be made of one or more of a variety of different materials (e.g., titanium, etc.). The mesh may be one or more of a variety of materials (e.g., palladium nickel, polyimide, etc.). After the fluid is pushed through the mesh, a droplet spray is formed and ejected through a mouthpiece port, carried by entrained air.

As shown in FIG. 5, the main body of a second example contains the vibrating member and transducer assembly 2603. The vibrating member and transducer assembly 2603 is encased by a vibrating member front cover 2602 and vibrating member rear cover 2604. The covers 2602, 2604 are held together by circular caps called the front and rear vibrating member cover holders 2605, 2608. The encased vibrating member is then put into the vibrating member enclosure 2601, followed by the vibrating member assembly spring 2606, and finally seated into the vibrating member device bracket 2607. The vibrating member enclosure allows the spring to press the vibrating member and transducer assembly to the membrane.

FIGS. 6A-6C illustrate an aperture plate assembly 1200 that includes an aperture plate 1216 (e.g., palladium-nickel) supported by a stainless-steel annulus 1218. The aperture plate is welded or bonded 1220 to the stainless-steel annulus 1218, thereby together allowing a thicker support material which is much less expensive than aperture plate material alone e.g., palladium-nickel. The stainless steel and aperture plate are bonded 1220 to the piezo-electric material 1222 wherein all the components form the aperture plate assembly 1200.

While the invention has been described with reference to exemplary embodiments, it will be understood by those skilled m the art that various changes may be made, and equivalents may be substituted for elements thereof without departing from the scope of the invention. In addition, many modifications may be made to adapt a particular situation or material to the teachings without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. An aerosol generation device comprising:
a reservoir configured to supply a volume of fluid;
a mesh in fluid communication with the reservoir and operably coupled to a vibrating member;
an electronic transducer coupled to the vibrating member, the electronic transducer configured to operate at a first frequency that oscillates the vibrating member to generate an ejected stream of droplets through the mesh; and
a circuit board or microcontroller providing a plurality of tuning signal waves to the electronic transducer during a tuning mode and measuring a current provided at a constant voltage; and
wherein the circuit board or microcontroller determines a detected resonance frequency at which a highest current draw is detected and selects the detected resonance frequency at the highest current draw to drive the electronic transducer.

2. The aerosol generation device of claim 1 further comprising a membrane coupled between the vibrating member and mesh.

3. The aerosol generation device of claim 2 wherein the aerosol generation device is a droplet delivery device including a mouthpiece or nosepiece configured for inhalation.

4. The droplet delivery device of claim 3, wherein the detected resonance frequency has a lower step size than the first frequency.

5. The droplet delivery device of claim 1, wherein the detected resonance frequency has a lower step size than the first frequency.

6. An aerosol generation device comprising:
a piezoelectric transducer operable to generate a droplet from fluid in the device;
an ejector mechanism coupled to the piezoelectric transducer and configured to receive the fluid; and
an auto-tuning circuit or microcontroller configured to adjust a first driving frequency of the piezoelectric transducer to a second driving frequency of the piezoelectric transducer to compensate for resonant frequency drift.

7. The aerosol generation device of claim 6 further comprising the auto-tuning or microcontroller configured to conduct current analysis on the piezoelectric transducer during operation and providing adjustment to the second driving frequency based on measurement of a highest current draw across various frequencies tested during the current analysis.

8. The aerosol generation device of claim 7 wherein the ejector mechanism includes an aperture plate coupled to the piezoelectric transducer.

9. The aerosol generation device of claim 8, wherein the aerosol generation device is a droplet delivery device including a mouthpiece or nosepiece configured for inhalation.

10. The droplet delivery device of claim 9, wherein the second driving frequency has a lower step size than the first driving frequency.

11. The aerosol generation device of claim 6, wherein the ejector mechanism includes an aperture plate coupled to the piezoelectric transducer.

12. The aerosol generation device of claim 11, further comprising a membrane coupled between the piezoelectric transducer and the aperture plate.

13. The aerosol generation device of claim 12, wherein the aerosol generation device is a droplet delivery device including a mouthpiece or nosepiece configured for inhalation.

14. The aerosol generation device of claim 8, further comprising a membrane coupled between the piezoelectric transducer and the aperture plate.

15. An aerosol generation device comprising:
a reservoir configured to supply a volume of fluid to a mesh of an ejector mechanism;
an electronic transducer operable to operate at a first frequency based upon a signal wave generated by a circuit, wherein the signal wave has a step size smaller than about 200 Hz, and wherein the transducer is operable to vibrate a mesh and generate at least one droplet from fluid supplied by the reservoir to the mesh;
a microcontroller or circuit that provides a plurality of signal waves to the electronic transducer during a tuning mode and measuring a current provided at a constant voltage, determines a detected resonance frequency at which a highest current draw is detected from providing the plurality of signal waves, and selects the detected resonance frequency to drive the electronic transducer.

16. The aerosol generation device of claim 15, wherein the electronic transducer is a piezoelectric transducer.

17. The aerosol generation device of claim 16, wherein the aerosol generation device is a droplet delivery device including a mouthpiece or nosepiece configured for inhalation.

18. The aerosol generation device of claim 17, further comprising a membrane coupled between the piezoelectric transducer and the aperture plate.

19. The aerosol generation device of claim 16, further comprising a membrane coupled between the piezoelectric transducer and the mesh.

20. The aerosol generation device of claim 15, further comprising a membrane coupled between the piezoelectric transducer and the mesh.
